(19) United States
(12) Patent Application Publication
(10) Pub. No.: US 2020/0054567 A1
(43) Pub. Date: Feb. 20, 2020

(54) DRUG DELIVERY SYSTEMS AND METHODS FOR PREPARATION THEREOF

(71) Applicant: NOVA SOUTHEASTERN UNIVERSITY, Fort Lauderdale, FL (US)

(72) Inventor: Enrique A. Nieves-Vazquez, Fort Lauderdale, FL (US)

(73) Assignee: NOVA SOUTHEASTERN UNIVERSITY, Fort Lauderdale, FL (US)

(21) Appl. No.: 16/347,219

(22) PCT Filed: Nov. 6, 2017
(86) PCT No.: PCT/US2017/060135

§ 371 Date: May 3, 2019

Related U.S. Application Data
(60) Provisional application No. 62/417,378, filed on Nov. 4, 2016.

(51) Int. Cl. A61K 9/20; A61K 31/4422; A61K 31/573; A61K 31/198; A61K 9/28

(52) U.S. Cl. CPC A61K 9/2072; A61K 31/4422; A61K 31/573; A61K 31/198; A61K 9/2031; A61K 9/2866; A61K 9/2873; A61K 9/2095; A61K 9/284

(57) ABSTRACT

The invention provides a drug delivery system in which a drug or another active substance is delivered from the surface of an inactive, placebo carrier. The system uses a placebo tablet carrier having a concavity or multiple concavities in the top surface for receiving a drug. After manufacture of the placebo tablet carrier, a dosage of the drug in liquid or semisolid form is deposited in the concavity and solidifies as a dot or disk on the tablet surface. Thus, the drug is carried on the surface of the tablet and is not part of the tablet bulk. The drug delivery system is particularly useful for delivery of low dose (i.e. potent) drugs, for delivery of multiple doses of a drug, or for delivery of multiple types of drugs. Additionally, the invention provides methods for preparation of the inventive drug delivery systems.

### FIELD OF THE INVENTION

The invention generally relates to drug delivery systems, particularly to improvements in drug delivery systems in which drugs are primarily delivered from the surface of an inactive, placebo carrier, and most particularly to a drug delivery system including a compressed, preferably inactive tablet having at least one concavity in a top surface for receiving a drug or other active substance and a drug or active substance deposited in the concavity.

### BACKGROUND OF THE INVENTION

The compressed tablet is one of the most common solid dosage forms in current pharmaceutical use. Generally, the tablet includes the active pharmaceutical ingredient (API) mixed together and compressed with various excipients that can act as both carriers for the drug and enhancers of therapeutic efficacy of the drug.

Conventional processing of solid drugs is a complex, multi-step process including granulation (via dry or wet methods), sifting, mixing, and milling followed by compression into tablets. These solid form processing steps are dusty operations and require expensive dust containment technology to minimize the environmental and occupational safety hazard it presents as the processing room and environmental air become contaminated with particulate matter. Further, some quantity of the drug is lost in the traditional process which thus requires the addition of an overage of drug to the formulation to compensate for the loss. As a result, manufacturing costs are increased.

Considering that drug delivery via compressed tablet is important in health systems worldwide, a drug delivery method which reduces or eliminates the above problems would be advantageous.

### SUMMARY OF THE INVENTION

In one embodiment, the invention provides a drug delivery system in which at least one drug or another active substance is delivered from the surface of an inactive, placebo carrier. The system uses a placebo tablet carrier having a concavity or multiple concavities in the top surface for receiving a drug. The drug delivery system is particularly useful for delivery of low dose (i.e. potent) drugs, for delivery of multiple doses of a drug, or for delivery of multiple types of drugs.

This invention represents a new concept of tablet formulation for drugs, particularly low dose (i.e. potent) drugs, which provides numerous benefits to pharmaceutical manufacturing, such as reduced manufacturing complexity, elimination of particulate contamination from powdered drugs, reduced cycle times, and decreased manufacturing costs, all leading to an overall improvement in quality in both the drug and in the manufacturing process. One preferred aspect of this inventive concept is dose delivery from a concentrated matrix deposited in a concavity of a compressed, placebo carrier tablet. By “placebo” it is meant that the tablet alone has no pharmacological effect for the recipient. The recipient refers to a subject or patient which can be a human or an animal to whom the tablet is administered or to whom the tablet can be administered. The placebo tablet is also referred to as a tablet carrier, core tablet, or placebo tablet carrier.

One aspect of the inventive method expands on the conventional practice of delivering drugs in tablet form by utilizing delivery of the active substance in a more concentrated form than that currently used for immediate release tablets. By “immediate release tablets” it is meant those tablets which exhibit no control of release; they dissolve or disintegrate rapidly to release the active substance. The terms “active ingredient”, “active substance”, “medicament”, “biologically-active agent”, and “active pharmaceutical ingredient (API)” all refer to the therapeutic entity or drug to be delivered to a recipient.

The dose of drug may be deposited on the tablet in the form of semi-dried minidisks or pellets. The disks may be made prior to deposition onto the tablet and may be deposited in solid form (disk shaped or pellet shaped) using a special binding agent on the surface.

The concentrated drug is placed on the surface of a tablet in a specially-designed formulation and is not integrated within the bulk of the tablet. In at least some embodiments, the tablet itself is a placebo tablet and serves only as a carrier for the drug/intended dose.

One aspect of the inventive concept represents a unique drug delivery method in which the drug is specifically formulated for insertion/deposit on the tablet surface in a spot as a liquid drop or spray to form a dot or disk that is easily identifiable by the unaided eye and analytical instruments. In preparing the delivery system, the drug is formulated in a solution, suspension, gel, or similar semi-solid and liquid forms to be deposited as a tiny drop melt on the tablet surface that will dry or solidify and stick to the tablet surface on contact. The tablet has a marked concavity in the center that will accommodate the volume of drug applied. The volume of the drop melt may exceed that of the concavity and may spread over and/or extend through the surface of the tablet to accommodate a larger load if necessary. After application and solidification of the drug, the tablet can be coated to provide a barrier against the environment. The tablet is then administered as a whole and is not split into parts.

The invention has many advantages and affords benefits to the patients, to the manufacturing industry, and to society.

Benefits to the Patient:

The drug delivery system provides a more accurate dose and less variation between doses than the conventional bulk tablets. The tablet size can be easily minimized, particularly for potent drugs, to increase patient comfort and compliance. Titration dose tablets are also feasible with this delivery system to provide individualized doses not available in the market. These doses may be prepared by a pharmaceutical compounder in a pharmacy or by a small-scale manufacturer. For example, a dose of 12 mcg of levothyroxine titration tablets allows a patient on a 25 mcg dose to increase it to 37 mcg which is not normally available in the market (the next dose is normally 50 mcg). The same applies to a patient on a 150 mcg dose where the next dose is 175 mcg. The dosage form according to the present invention allows preparation of titration tablets more easily than the traditional method.

Benefits to the Manufacturing Industry:

The drug delivery system will dramatically reduce the cost of producing a tablet since in certain embodiments all tablets will be placebos and the cleaning validation efforts as well as the potential for contamination will be greatly minimized. The drug spot/disk will be identifiable with unaided eye and by analytical instruments which will allow for implementation of Process Analytical Technologies (PAT) at the manufacturing line. Thus, close to 100% of the tablets produced may be inspected for drug identification and content.

Process Analytical Technologies (PAT) were initiated by the Food and Drug Administration (FDA) to promote comprehensive quality assurance monitoring throughout the pharmaceutical industry. PAT can be defined as a system for designing, analyzing, and controlling manufacturing through timely measurements (i.e. during processing) of critical quality and performance attributes of raw and in process materials, and processes with the goal of ensuring final pharmaceutical product quality. See U.S. Pat. No. 8,645,084 B1.

Since the drug is formulated in a liquid form, there is no drug powder produced during manufacture and thus, particulate contamination of the environment is avoided.

There is no in-process analytical testing for drug content during the tableting process and further the cleaning validation process for compressed tablets is eliminated.

Stability Enhancement:

the stability profile of the drug may be enhanced by the lower excipient-drug ratio used in the droplet or disk as compared to traditional formulations, and by the simplicity of the formulation (i.e. preferably, but not limited to, no more than three ingredients) which minimizes the probability of interaction between drug and excipients. Also drugs sensitive to oxygen or moisture are protected by the engulfment of the drug inside the polymer (could be hydrophobic or hydrophilic) which minimizes contact between drug and the environment.

Benefits to Society:

perhaps the greatest benefit to society from this drug delivery system is the cost reduction. When the costs of tablet production decrease, the costs of the medication will also decrease.

In a general aspect, the invention provides tablets for a drug delivery system.

In a general aspect, the invention provides a drug delivery system.

In a general aspect, the invention provides a method for preparing a drug delivery system.

In a general aspect, the invention provides a method for delivering a drug from the surface of a tablet.

In one aspect, the invention provides a tablet formulation for low dose (i.e. potent) drugs. “Low dose” drugs are those having a dose of about 25 mg or lower. The term “about” in this context refers to amounts near or close to 25 mg which can be formulated and carried by the inventive tablet. At this time, CarreTab™ is the intended commercial name of this inventive tablet.

In another aspect, the invention provides a compressed, inactive tablet for carrying an active substance formulated for immediate release of the active substance (FIG. 1A). The tablet includes a concavity in a top surface thereof for receiving the active substance. The top surface of the tablet does not extend in height above edges of the concavity. This concavity is an indentation shaped to receive a predetermined volume of the active substance to be delivered and is indented to about 35% of the thickness of the tablet. This indentation or concavity supports the disk or dot of the active substance and thus is not and cannot be considered a hole extending throughout the entirety of the tablet. The term “about” in this context refers to amounts near or close to 35% which can reasonably be considered representative of an indentation or concavity penetrating through about 35% of the thickness of the tablet.

The placebo tablet carrier of the drug delivery system is shown in FIG. 1A. The arrow points to the concavity in the top surface of the tablet. This placebo tablet carrier having a drug or other active substance deposited in the concavity is shown in FIG. 1B.

In a further aspect, the invention provides a drug delivery system including the tablets described immediately above.

In another aspect, the invention provides a drug delivery system including a compressed, inactive tablet having a concavity in a top surface for receiving an active substance and an active substance deposited in the concavity. The tablet is formulated for immediate release of the active substance.

The active substance is a substance providing a desired pharmacological effect such as a drug or a low-dose drug. Several, non-limiting examples are steroidal hormones, synthetic hormones, and amlodipine besylate.

A non-limiting example of a steroidal hormone is the cortisone derivative, hydrocortisone. This medication is a corticosteroid used to treat symptoms of skin conditions such as itching, swelling, and rash. Many skin conditions can cause these symptoms; i.e. allergies, eczema, insect bites, poison ivy/oak, and dermatitis. Hydrocortisone is available in a variety of forms, with and without a prescription from a doctor. This and other information regarding hydrocortisone can be found at the website WebMD.

A non-limiting example of a synthetic hormone is levothyroxine. This medication replaces or provides additional thyroid hormone and is used to treat thyroid conditions such as hypothyroidism, goiters, and thyroid cancers. Levothyroxine is available in different brands with a prescription from a doctor. This and other information regarding levothyroxine can be found at the website WebMD.

In addition to steroidal and synthetic hormones, amlodipine besylate is another low-dose drug that can be formulated according to the invention. Amlodipine besylate is a calcium channel blocker used to treat high blood pressure by relaxing blood vessels such that blood flows more easily. Amlodipine besylate is available with a prescription from a doctor. This and other information regarding amlodipine besylate can be found at the website WebMD.

In a further aspect, the invention provides drug delivery systems for hydrocortisone, levothyroxine, and amlodipine besylate.

In another aspect of the inventive drug delivery system, the tablet includes a pharmaceutically-acceptable coating for protecting the drug against the surrounding environment. Non-limiting examples of such pharmaceutically-acceptable coatings are hydroxypropyl methylcellulose (HPMC), polyvinylpyrrolidone (PVP), cellulose derivatives, starch derivatives, acrylates, acetates, gelatin, and shellac. A pharmaceutically-acceptable coating is inactive, has no biologic or therapeutic effect, and does no harm to the recipient of the medication.

An additional aspect of the invention provides a method for preparing a drug delivery system. The method includes the steps of selecting a drug for delivery; selecting a polymer for dispersion of the drug; preparing a liquid or semi-solid drug dispersion by dispersing the drug in the polymer; and depositing a droplet of the drug dispersion in a concavity in a top surface of a compressed, inactive tablet such that the droplet solidifies to form a disc on the top surface of the tablet.

Another aspect of the inventive method includes a step for selecting a temperature at which to deposit the droplet of the drug dispersion in the concavity in the top surface of the tablet. Deposition of the droplets at different temperatures affect the density of the droplet and the load per volume. Melting temperature of the polymer could reach 90° C. or higher, if needed (below decomposition temperature). Drug loading and dissolution in the polymer depends on drug tolerance but could be accomplished at 60°, 50°, or 40° C. as long as the polymer remains in melted form and relatively liquid to flow through the dispenser.

For example, fatty materials such as cocoa butter may be melted gently at 34° C. before dispersing the drug in it, and the drug may be incorporated at that temperature. Other polymers such as PEG blends may be melted at higher temperatures depending on the design of the blend as long as the temperature of the melt is maintained below the decomposition temperature of the polymer. In one case the polymer was melted and stirred at up to 90° C. Then it was allowed to cool at 60° C. with stirring to disperse the drug in it. Cooling and stirring continued until the drug is fully dispersed. After the drug is incorporated in the warm polymer, the liquid melt is ready for use in the concavity in the top surface of the tablet.

Non-limiting examples of polymers selected for dispersion of the drug include polyethylene glycol (PEG) mixtures of various molecular weights, mixtures of hydrogenated vegetable oils, polyoxyl stearate polymers, glyceryl monostearate and other pharmaceutical polymers. Other suitable non-limiting examples of polymers include the following.

Cellulose-Based Polymers: ethyl cellulose, carboxymethylcellulose, hydroxypropylmethylcellulose, cellulose acetate phthalate, hydroxyethylcellulose, and others.
Hydrocolloids: alginic acid, carrageenan, chitosan, hyaluronic acid, and pectinic acid.
Synthetic Polymers: polyethylene oxide, polyacrylic acid, polyvinyl pyrrolidone, poly vinyl alcohol, and polyacrylamide.
Starch-Based Polymers: starch, sodium starch glycolate, and starch glycerite.
Plastics: polyurethane and polycarbonate.
Other polymers can include, but are not limited to, traditional polymers used in pharmacy.

Upon administration, the drug droplet can partially fill the concavity, completely fill the concavity, or can overfill the concavity. In the instance of overfill, the drug droplet can spread over and/or extend through the surface of the tablet to accommodate a larger load than the volume of the concavity if necessary.

When preparing the inventive drug delivery system, various concentration ratios and drug loads can be used according to purpose. As exemplified in the experimental examples, an optimum amount of 1 to 20 mg per tablet was achieved with a top load of 10 mg without affecting the quality of the tablet. A range of active droplet volumes may be added to each tablet. As further exemplified in the experimental examples, a range of 0.25 to 10 uL is considered optimum for a volume of drug dispersion to be deposited, using a drug concentration of 33% (w/v). This volume however, depends on the size of the tablet and may be higher (for example, over 50% w/v).

Larger amounts of a drug can be accommodated by larger tablets. A non-limiting example is a dose limitation in a range of about 10 mg to about 20 mg in a tablet of 1000 mg in weight. The term “about” in this context refers to amounts near or close to an amount of the drug that can be successfully delivered by a tablet weighing 1000 mg.

In a further aspect, the invention provides a compressed, inactive tablet for carrying at least one active substance including a plurality of concavities in the surface for receiving the active substance; i.e., a compressed, inactive tablet having a concavity in a top surface (FIG. 6B) and a plurality of concavities spaced apart and embedded within the concavity in the top surface. The concavity in the top surface is also referred to as the “main concavity.” Each of the plurality of concavities is separated and spaced apart from the other concavities such that any substance deposited in the cavity is isolated from substances deposited in the other concavities. A preferred, albeit non-limiting, embodiment includes three concavities embedded within the top surface of the tablet. The tablet can be formulated as either a solid or a semi-solid and the top surface (of the tablet) does not extend in height above edges of the concavity (FIG. 6A). In one aspect, the concavity is an indentation shaped to receive a predetermined volume of the active substance to be delivered and is indented to about 35% of the thickness of the tablet. This indentation or concavity supports the disk or dot of the active substance and thus is not and cannot be considered a hole extending throughout the entirety of the tablet. The term “about” in this context refers to amounts near or close to 35% which can reasonably be considered representative of an indentation or concavity penetrating through about 35% of the thickness of the tablet. The ratios of diameters are key to tablet design. In one preferred, albeit non-limiting embodiment, the tablet has a diameter of about 8 mm and the concavity in the top surface has a diameter of about 3 mm. The term “about” in this context refers to diameters close to or near 8 mm (or 3 mm) at which the tablet retains the desired design.

In a further aspect, the compressed, inactive tablet also includes an identifier for facilitating identification of the tablets. Preferred, albeit non-limiting, examples are ink and an identifier embedded in ink. A very small amount of ink nanoparticles are injected onto the surface of the tablet and spread to act like a fingerprint facilitating detection of the tablet. Tablets can be individualized and tagged with these identifiers.

In a further aspect, the invention provides a drug delivery system including the tablets containing an identifier described immediately above.

In an alternative embodiment, the tablet is shaped as a bullet (FIG. 6C) having at least one curved end and an indentation for the concavity in the curved end or in a flat side. All embodiments of the described tablets can be shaped as bullets.

In a further aspect, the invention provides a drug delivery system including the tablets shaped as bullets described immediately above.

In yet another aspect, the invention provides a drug delivery system including a compressed, inactive tablet having a concavity in a top surface for receiving at least one active substance, a plurality of concavities spaced apart and embedded within the concavity in the top surface, and an active substance deposited in the concavity. Each of the plurality of concavities supports the active substance deposited in the concavity in the top surface. The tablet can be formulated either as a solid or as a semi-solid and (the tablet) can be formulated for immediate release of the active substance. The tablet can include a pharmaceutically-acceptable coating for protecting the drug against the surrounding environment as described above.

The active substance can be a drug or a low-dose drug. Several, non-limiting examples are steroidal hormones, synthetic hormones, and amlodipine besylate. A non-limiting example of a steroidal hormone is the cortisone derivative, hydrocortisone. A non-limiting example of a synthetic hormone is levothyroxine. In addition to steroidal and synthetic hormones, amlodipine besylate is another low-dose drug that can be formulated according to the invention. Hydrocortisone, levothyroxine, and amlodipine besylate are described above.

In a further aspect, the invention provides drug delivery systems for hydrocortisone, levothyroxine, and amlodipine besylate that utilize a tablet having a plurality of concavities spaced apart and embedded within the concavity in the top surface.

In another aspect of the inventive drug delivery system, the active substance, rather than being deposited in the main concavity in the top surface of the tablet, is deposited in one of the plurality of concavities embedded within the main concavity. In a particularly preferred embodiment, the tablet includes three concavities embedded within the main concavity and a drug as the active substance deposited in each of the three concavities. In one aspect of the preferred embodiment, the active substance in the three concavities can be three dosages of one drug, or alternatively can be three different drugs. In this delivery system, the drug can be water soluble or water insoluble and formulated as a solid, a semi-solid, an emulsion, a suspension, a solution, a gel, or a fatty depot. The volume of drug deposited ranges from about a few microliters (μl) to about 500 μl. The term “about” in this context refers to volumes at near 500 μl that can be successfully deposited within the concavity. Steroidal hormones, synthetic hormones, and amlodipine besylate can be delivered using this system as described above.

The inventive drug delivery systems are further contemplated for use in combination therapy. For combination therapy, the tablet, rather than functioning as an inactive carrier, is formulated to include at least one active substance, such as a drug. A non-limiting example of combination therapy includes levothyroxine formulated in the core tablet with liothyronine deposited in the concavities on the tablet surface in varying strengths.

In another embodiment, the inventive drug delivery system provides tablets that discourage counterfeiting of the tablet by carrying the active substance in a tablet logo (FIGS. 5A-E).

In an aspect of the anti-counterfeiting embodiment, the inventive drug delivery system includes a molded tablet having a design or logo embossed thereon rather than concavities. The active ingredient or drug is formulated with the design material and applied to the tablet with application of the design; i.e. drug in print as an identifier embedded in ink.

In another aspect of the anti-counterfeiting embodiment, the inventive drug delivery system includes a tablet having a design or logo and an active substance embossed on the surface of the tablet. The design is formed from a mixture of an identifier, such as, but not limited to ink, and the active substance, such as, but not limited to, a drug.

The invention additionally encompasses a method for delivering at least one active substance, such as, but not limited to, a drug, to a subject in need thereof from a surface of a tablet carrier including the steps of providing a compressed tablet (active or inactive) having a concavity in a top surface for receiving at least one active substance and at least one active substance deposited in the concavity and administering the tablet to the subject in need thereof.

In yet another aspect, the invention includes tablets made in various forms such as, but not limited to, round, elongated (capsule-shaped), bullet-shaped, and boat-shaped. The tablets may contain flavoring agents such as, for example chocolate, cherry and other flavors, sweeteners, and they may be made as a chewable tablet or a swallowable tablet.

All of the tablets described herein can be used in any of the drug delivery systems described herein.

Other objectives and advantages of this invention will become apparent from the following description taken in conjunction with the accompanying drawings, wherein are set forth, by way of illustration and example, certain embodiments of this invention. The drawings constitute a part of this specification and include exemplary embodiments of the present invention and illustrate various objects and features thereof.

### BRIEF DESCRIPTION OF THE DRAWINGS

A more complete understanding of the present invention may be obtained by references to the accompanying drawings when considered in conjunction with the subsequent detailed description. The embodiments illustrated in the drawings are intended only to exemplify the invention and should not be construed as limiting the invention to the illustrated embodiments.

FIG. 1A shows one embodiment of the placebo tablet carrier of the drug delivery system. The arrow points to the concavity for receiving an active substance, such as a drug, in the top surface of the tablet.

FIG. 1B shows the placebo tablet carrier of FIG. 1A having a drug or other active substance deposited in the concavity.

FIG. 2A is an infrared spectra obtained using Fourier Transform Infrared Spectroscopy (FTIR) of hydrocortisone embedded in polyethylene glycol (PEG) as deposited in the concavity of a placebo tablet carrier.

FIG. 2B is an FTIR spectra of polyethylene glycol (PEG) alone deposited in the concavity of a placebo tablet carrier for comparison with the spectra shown in FIG. 2A.

FIG. 2C is an FTIR spectra of pure hydrocortisone for comparison with the spectra shown in FIGS. 2A and 2B.

FIG. 3A is an infrared spectra obtained using Fourier Transform Infrared Spectroscopy (FTIR) of amlodipine besylate embedded in polyethylene glycol (PEG) as deposited in the concavity of a placebo tablet carrier.

FIG. 3B is an FTIR spectra of polyethylene glycol (PEG) alone deposited in the concavity of a placebo tablet carrier for comparison with the spectra shown in FIG. 3A.

FIG. 3C is an FTIR spectra of the polyethylene glycol (PEG) blend (unmelted) for comparison with the spectra shown in FIGS. 3A and 3B.

FIG. 4 is an infrared spectra obtained using Fourier Transform Infrared Spectroscopy (FTIR) of levothyroxine embedded in polyethylene glycol (PEG) as deposited in the concavity of a placebo tablet carrier. This spectra is superimposed with the FTIR spectra of polyethylene glycol (PEG) alone deposited in the concavity of a placebo tablet carrier (FIG. 5B). The region of 1550 to 1900 cm⁻¹ and the peak at 2400 cm⁻¹ could be used for Process Analytical Technology (PAT) detection in a manufacturing plant.

FIGS. 5A-E are sketches showing multiple views of an alternative embodiment of the placebo tablet carrier of the drug delivery system. This embodiment is a round tablet having a main concavity in the top surface and three smaller concavities spaced apart and embedded within the main concavity. FIGS. 5A-B show views of the top surface of the tablet including the concavities; FIG. 5C shows a side view of the tablet; and FIGS. 5D-E show views of the bottom surface of the tablet showing an embossed brand logo or other identification marking the tablet.

FIG. 6A is a sketch showing a side view of the placebo tablet carrier of FIG. 1A. The indentation represents the concavity in the center of the top surface of the tablet.

FIG. 6B is a sketch showing a top view of the placebo tablet carrier of FIG. 6A FIG. 6C is a sketch showing a side view of another embodiment of the placebo tablet carrier having a bullet-like shape with a rounded end and a flat end. In this embodiment of the tablet, a concavity can be embedded in the curved surface of the top end or in a flat side surface.

FIG. 7 is a sketch showing top and side views with various dimensions identified, including the diameter of the tablet (D) and the diameter of the indentation or concavity (d). A preferred ratio of D to d is 8:3.

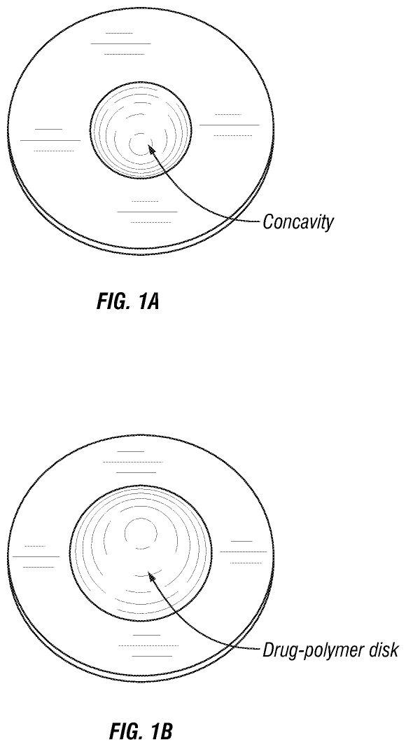

### DETAILED DESCRIPTION OF THE INVENTION

For the purpose of promoting an understanding of the principles of the invention, reference will now be made to embodiments illustrated herein and specific language will be used to describe the same. It will nevertheless be understood that no limitation of the scope of the invention is thereby intended. Any alterations and further modification in the described drug delivery systems, compositions, tablets, formulations, and methods and any further application of the principles of the invention as described herein, are contemplated as would normally occur to one skilled in the art to which the invention relates.

In one embodiment, the drug delivery system described herein represents a new dosage form having a placebo tablet that carries the load of active drug in a specific spot on the surface such that the active drug is delivered from the surface of the tablet. The spot is identified by a concavity on the upper or top surface of the tablet that serves to accommodate the drug load in the form of a semi-solid droplet or disk. In an industrial setting, tablet manufacturing of potent or low dose drugs (i.e. steroids) is simplified in this manner as the core tablets are made as placebo tablets. The active ingredient/substance or drug is added in liquid form at the end of the manufacturing process to the core tablet. The active substance/ingredient adheres to the surface of the tablet and is not formulated as part of the bulk of the tablet. Thus, the same core tablet formulation may be used with many different drugs and/or other active substances (e.g. vitamins).

Additionally, this drug delivery minimizes cleaning steps and change overs during tablet processing. Modern liquid technologies allow for the delivery of precise micro-volumes which help design drug delivery systems of low dose drugs with high accuracy and precision.

Conventional processing of solid drugs includes steps for granulation, sifting, mixing, and milling followed by compression into tablets. These solid form processing steps create drug powder containment problems and represent an environmental and occupational safety hazard as the processing room and environmental air become contaminated with particulate matter. The inventive manufacturing process helps contain the drug as it will be processed in a liquid phase and not in the solid state. The active ingredient/substance or drug would be transferred from the raw material stage directly to the wet phase containing the dispersant polymer.

The inventive manufacturing process includes dispersing the active ingredient/substance or drug in a suitable polymer or a blend thereof. The solubility of the drug in water determines the choice of molecular weight polymers that are used for the polymer blend. Polyethylene glycol (PEG) is commonly used. For polymers that require heat, the drug should be dispersed in the melted polymer at a temperature that will not affect the drug. The goal is to engulf the drug with melted polymer which will cool and solidify rapidly when placed in the concavity of the tablet which serves to hold the droplet of the dispersion while in the liquid phase. The dispersion solidifies as a disc or dot and adheres to the tablet with enough strength to tolerate average handling. The solidified dispersion and the placebo tablets should be stored at room temperatures below 25° C. to prevent melting. Polymers of higher melting points may be used to make the final product more tolerant to warm environments and may not require refrigeration for storage.

The drug-loaded tablet may be coated with a compatible polymer such as hydroxypropyl methylcellulose (HPMC), polyvinylpyrrolidone (PVP), cellulose derivatives, starch derivatives, acrylates, acetates, gelatin, shellac, and other pharmaceutically-acceptable coatings to add physical protection to the drug dispersion.

Formulations of steroidal hormones, synthetic hormones, and other low dose drugs, i.e. lower than 25 mg, are ideal for the inventive method as exemplified in the examples presented below.

### Example 1

### Droplet Composition and Formulation of Hydrocortisone

Hydrocortisone is a steroidal hormone. Due to its similarity in molecular structure with other hormones, it (hydrocortisone) can be used as a model for all steroidal drug hormones available in the market and potentially usable in the inventive drug delivery system.

A dispersion of hydrocortisone USP was prepared using a concentrated dispersion of the drug in a polyethylene glycol (PEG) blend. PEG of various molecular weights may be used in the PEG mix/blend. One (1) gram of drug was dispersed in 2 ml of melted PEG mix/blend to provide a 33% load of the drug in the dispersion. Various concentration ratios and drug loads can be used according to purpose. An optimum amount of 1 to 5 mg per tablet was achieved with a top load of 10 mg without affecting the quality of the tablet. Deposition of the droplets at different temperatures affect the density of the droplet and the load per volume. A range of active droplet volumes may be added to each tablet. A range of 0.25 to 10 uL is considered optimum for a volume of drug dispersion to be deposited, using a drug concentration of 33% (w/v).

Exemplary drug delivery systems of this embodiment include tablets formulated with 1 mg hydrocortisone (designation drop 3.1 mg; total table weight 247.4 mg) and tablets formulated with 3.9 mg hydrocortisone (designation drop 11.6 mg; total table weight 257.8 mg). The designation drop is the amount of melted polymer added.

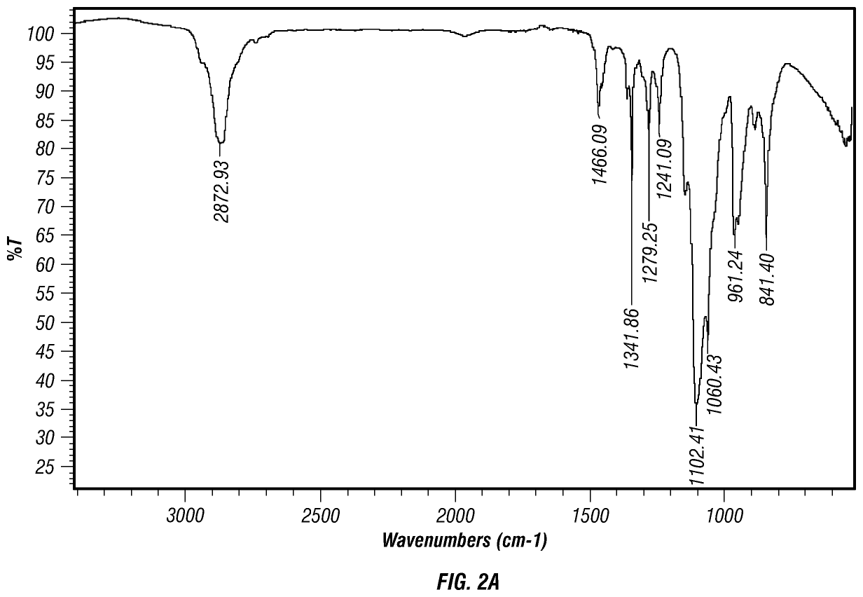

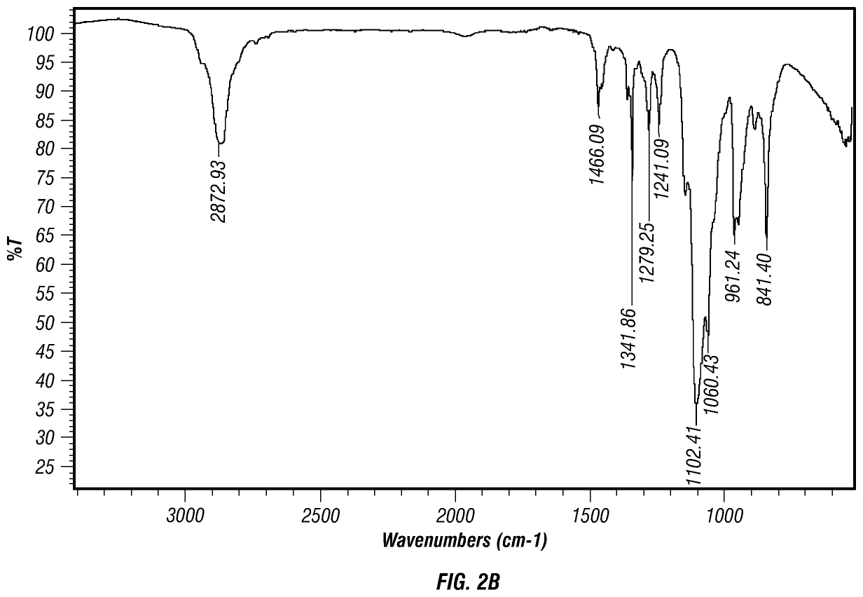

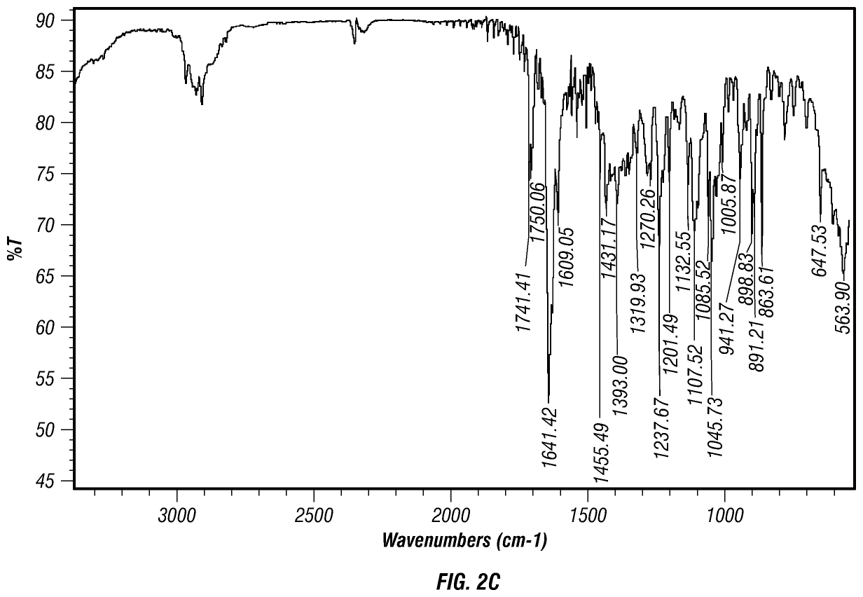

An infrared spectra obtained using Fourier Transform Infrared Spectroscopy (FTIR) of hydrocortisone embedded in polyethylene glycol (PEG) as deposited in the concavity of a placebo tablet carrier is shown in FIG. 2A. An FTIR spectra of polyethylene glycol (PEG) alone deposited in the concavity of a placebo tablet carrier for comparison to the infrared spectra of hydrocortisone embedded in PEG is shown in FIG. 2B. From the similarity of these two spectra, it appears that the instrument detects only the polymer. The drug, i.e. hydrocortisone, is embedded within the polymer structure. An FTIR spectra of pure hydrocortisone for comparison (with the spectra shown in FIGS. 2A and 2B) is shown in FIG. 2C.

### Example 2

### Droplet Composition and Formulation of Amlodipine Besylate

Amlodipine besylate is another representative example of a group of drugs that may be formulated in the inventive drug delivery system. It can be prepared in semi-solid form using a polymer blend of two or more polyethylene glycol (PEG) polymers of different molecular weights. Amlodipine besylate was prepared as above in Example 1. The dispersion can be diluted or concentrated further to facilitate deposition onto the tablet or to achieve a desired concentration of drug or to adjust the drug content per droplet.

An exemplary mixture/blend of PEG 400, PEG 8000, and Polysorbate 80 can be used to dissolve the drug in the melted phase. Other polymeric materials such as, but not limited to, fatty bases can also be used.

A PEG/drug droplet placed on top of the placebo tablet forms a mini-disk or dot upon drying or solidifying. The PEG/drug droplet changes color from beige to yellow depending on the time it is heated.

Exemplary drug delivery systems of this embodiment include tablets formulated with amlodipine besylate in amounts of 0.8 mg, 5.33 mg, and 6 mg (total polymer-drug solidified disks weigh 2.4 mg, 16 mg, and 18 mg, respectively; total tablet weight 240 mg).

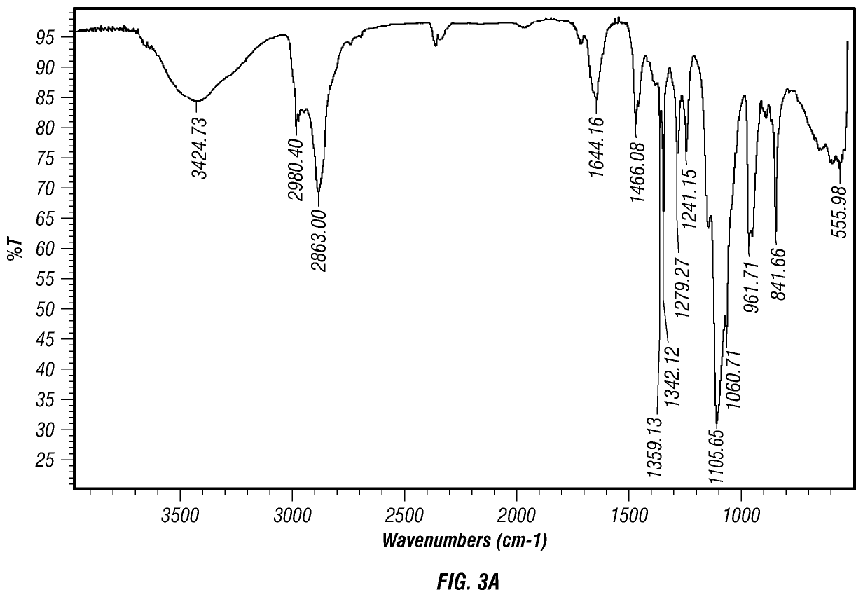

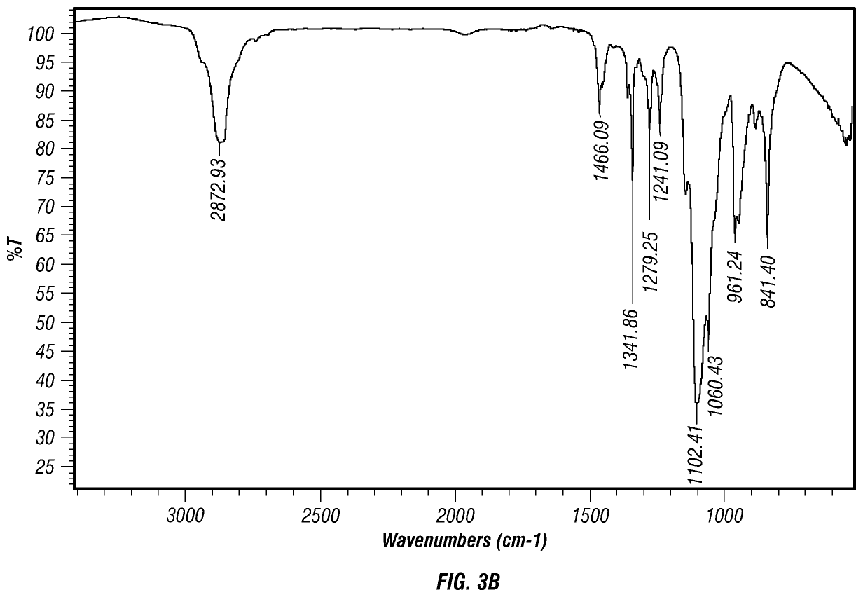

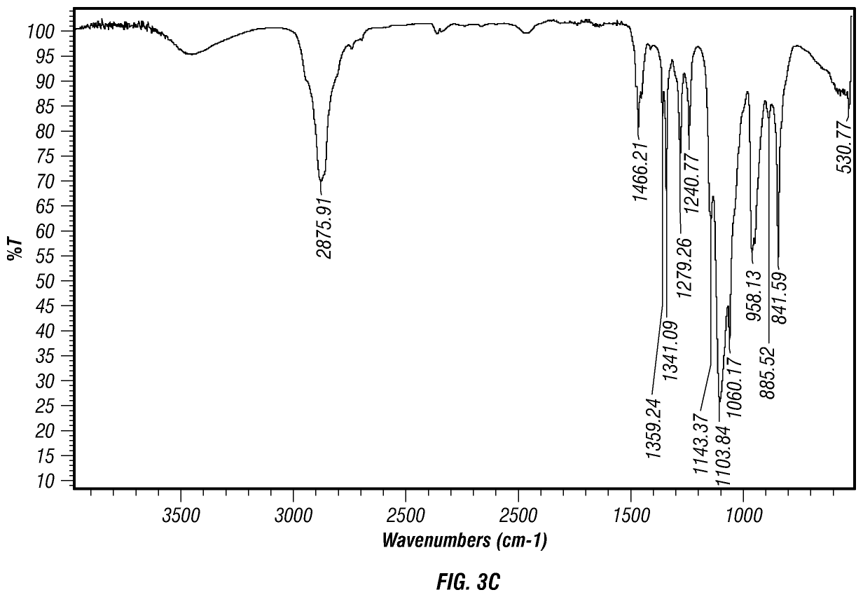

An infrared spectra obtained using Fourier Transform Infrared Spectroscopy (FTIR) of amlodipine besylate embedded in polyethylene glycol (PEG) as deposited in the concavity of a placebo tablet carrier is shown in FIG. 3A. An FTIR spectra of polyethylene glycol (PEG) alone deposited in the concavity of a placebo tablet carrier for comparison with the spectra of amlodipine besylate embedded in polyethylene glycol (PEG) is shown in FIG. 3B. The amlodipine besylate spectra versus the PEG placebo spectra shows some differentiation which can be employed for Process Analytical Technology (PAT) detection in a manufacturing plant. An FTIR spectra of the polyethylene glycol (PEG) blend, un-melted, for comparison (with the spectra shown in FIGS. 3A and 3B) is shown in FIG. 3C.

### Example 3

### Droplet Composition and Formulation of Levothyroxine

Levothyroxine is a synthetic hormone used in the form of tablets to treat thyroid deficiency. The dose ranges from 25 mcg to 300 mcg per tablet and is commercially-available in 12 different strengths. The inventive formulation allows the drug to be engulfed by a liquid polymer prior to its deposition onto a tablet. This way the molecules are not subject to the forces involved in the manufacture of tablets which can improve consistency of dose and stability. Levothyroxine was prepared as above in Example 1. All 12 strengths of levothyroxine can be formulated for the inventive drug delivery system.

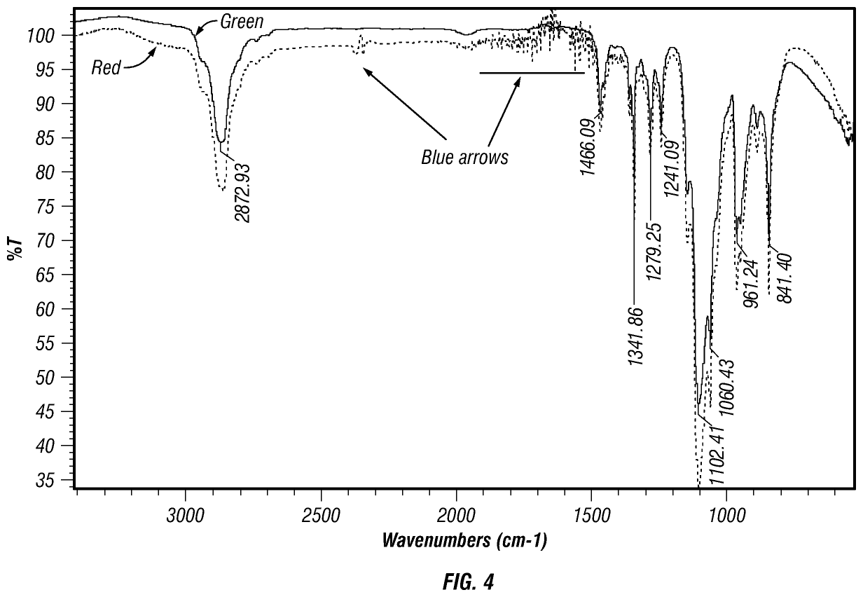

An infrared spectra obtained using Fourier Transform Infrared Spectroscopy (FTIR) of levothyroxine embedded in polyethylene glycol (PEG) as deposited in the concavity of a placebo tablet carrier is shown in FIG. 4. This spectra is superimposed with the FTIR spectra of polyethylene glycol (PEG) alone deposited in the concavity of a placebo tablet carrier (FIG. 3C). The region of 1550 to 1900 cm⁻¹ and the peak at 2400 cm⁻¹ could be used for Process Analytical Technology (PAT) detection in a manufacturing plant.

### Example 4

### Multi-Dosage Drug/Multi-Drug Delivery System

CarreTab Multi-Disk Technology or CTMD is the intended commercial name for the embodiment of the inventive drug delivery system involving multiple drug dosages and/or multiple drugs. Aspects of this embodiment encompass: 1) delivery of three different doses of a drug from the surface of a carrier tablet, and 2) delivery of three different drugs from the surface of a carrier tablet. There is no contact between the drug dosages/drugs on the surface of the tablet. Thus, physico-chemical interactions within the dosage forms/different drugs is minimized.

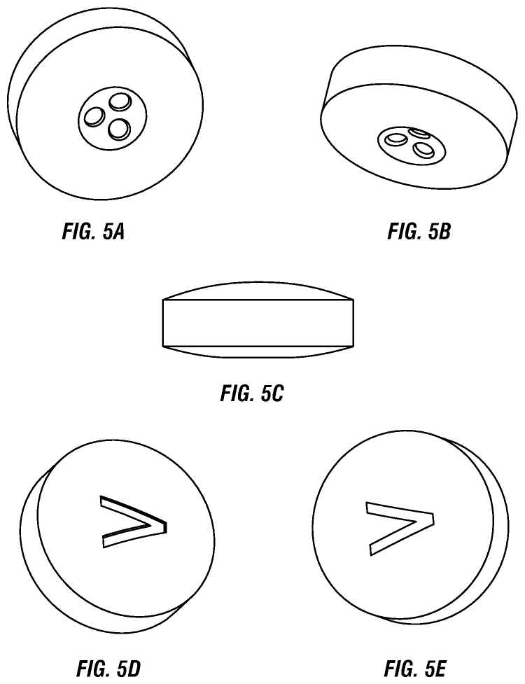

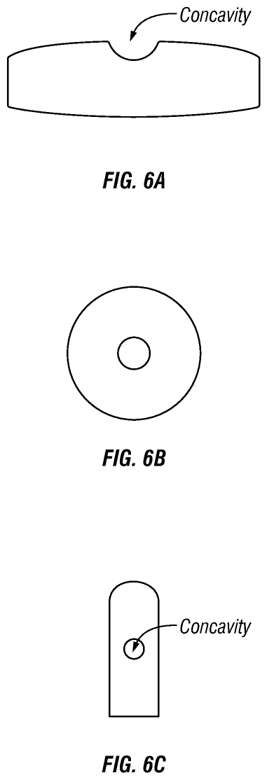

The dosage form is prepared as two separate and distinct phases or forms; i.e. a core tablet and the semi-solid minidisks with drug that fill the concavities in the core tablet (FIGS. 5A-E). The different phases are joined together as one dosage form of two or more compartments where one compartment (core tablet) serves as the main carrier (placebo portion) and the other compartment (semi-solid) serves as the active (drug-containing) portion.

Minidisks containing the drugs are located in three or more concavities that themselves are embedded within a main bigger concavity on the surface of the carrier/core tablets (FIGS. 5A-E). The minidisks have a volume that ranges from a few microliters (μl) to close to (but not limited to) approximately 500 microliters. The concavities are functional since they support and hold drugs (minidisks) properly formulated for this type of delivery application. Further, other functionalities of the concavities include: serving as base to strengthen the bond between drug formulation and core tablet, separating the different doses of a drug or different active ingredients, serving as ornamentation and/or decoration, and serving as an indicator or identifier of a specific tablet. The drug-containing concavities could be coated with polymer or suitable pharmaceutical agents to add stability to the dosage form. This CTMD technology allows for multiple detection of different drugs simultaneously by remote spectrophotometry at line since each drug is separately concentrated in a specific spot on the surface of the tablet main concavity.

### Core Tablet as an Inactive, Placebo Carrier

The core tablet is prepared, as described above, in a traditional way via direct compression, dry granulation or wet granulation methods, and formulated as a placebo. The core tablet is prepared with a hole or main concavity on the surface specifically designed to carry a dose of a drug in the form of mini disks or semi-solid depots. Within this concavity there are three or more mini-concavities that hold the mini-disks in place. Or alternatively, the mini-concavities serve as footing to hold a larger disk or depot in the whole main concavity. The core tablet is preferably prepared as a compressed tablet, but may be prepared as any pharmaceutical table suitable, for example, but not limited to a molded tablet. The drug depot (semi-solid mini-disks) would be prepared separately from the core tablet and placed in the selected spot on the surface of a tablet to combine or fuse the two phases.

### Tooling Design of the Core/Carrier Tablets

A logo can be embossed on one side of the tooling (lower punch) and indentations for making the concavities on the other side of the tooling (upper punch). See FIGS. 5A-E. Tapered dies can be used to relieve pressure during compression cycles.

The ratios of diameters are important to the tablet design to minimize breaking the tooling during processing and (the ratios) also provide desired concavity and mechanical strength to the finished tablets. If a capsule or bullet shape is used instead of a tablet design, the diameter could be referred to as width, regardless of whether a circular cross-section is present.

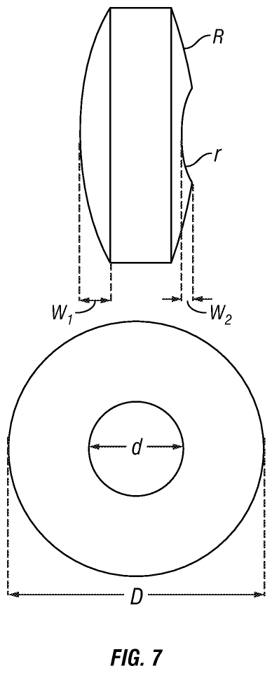

Regarding relative measurements for tooling design, Fibonacci ratios (0.610-0.67) can be used as starting point or guideline between main concavity diameter and tablet diameter, between small concavities and main concavity, and between thickness of the concavities and thickness of the tablet, but these ratios may vary beyond the region specified above. See FIG. 7 in which D refers to the tablet diameter; d refers to the concavity diameter; R refers to the radius of curvature of the tablet; r refers to the radius of curvature of the concavity; W₁ refers to the depth of the crown of the tablet; and W₂ refers to the depth of the concavity. For example, a core tablet with a diameter of 8 mm would have a main concavity diameter of 3 mm (which would leave a difference of 5 mm from the inner circle to the edge of the tablet (these three numbers follow the Fibonacci sequence in the series (e.g. 1, 1, 2, 3, 5, 8, etc.). Similarly, if the thickness of the tablet is 4 mm, the depth of the concavities should not exceed 1.5 mm to maintain the Fibonacci ratios as much as possible even though 1.5 and 4 are not Fibonacci numbers.

### Preparation of Drug/Active Disks

The active minidisks or semisolid formulations may be prepared as described above.

The minidisks or drug depot can be formulated as an emulsion or suspension (with particle sizes in the nano-meter and micro-meter levels), as a solution, gel, fatty depot, semi-solid, or solid. This allows formulation of both water soluble and water insoluble drugs.

With semi-solid and solid formulations, the drug is prepared in high concentration in a hydrophobic melt (made of PEG mixtures, fatty base or similar) and placed on the core tablet surface as a drop or spray depot where it will solidify at room temperature. The volume of the depot is in the microliter range.

With emulsions, suspensions, and solutions, the drug is formulated in a very high concentration and the solvent is allowed to evaporate during application of the depot via spraying or similar solvent evaporation method.

### Core Tablet as an Active Tablet

In addition to drugs on the surface, the core tablet may contain a drug to produce a combination product. In this aspect, the core tablet is prepared in the traditional way as above but further includes an active drug. It would be used in the inventive systems when the administration of two drugs or more is desired. This tablet can be used to accommodate one drug in two or more doses, or two or more drugs in one dosage form. It can be used to deliver two doses of the same drug with different release properties, to deliver two different drugs (one in the bulk tablet and the other on the surface as a semi-solid), or more than two drugs (for example more than one drug depot may be placed on the core tablet). This way the drugs are located in different compartments in the same system and thus could be formulated differently. Further, the system may contain two drugs inside the core tablet and a third drug on the surface semi-solid portion.

### Versatility of the Multi-Dosage Drug/Multi-Drug Delivery System

The CTMD system is a drug delivery system that allows three or more different drugs or formulations of the same drug in the same system separated by different compartments to minimize physical contact and eliminate the potential of chemical interaction within the dosage form. The formulations in the CTMD can differ in drug identity, molecular size, dose, amount, concentration, composition, excipients, release profiles (dissolution and absorption), and methods of manufacture. The CTMD system may contain more than two drugs without interacting due to the separation of compartments and technologies in the same tablet device.

The drugs in the CTMD system may be identifiable by analytical instruments for PAT (Process Analytical Technologies) applications. If desired, instead of a drug, an identifier (a secret chemical) may be used instead of a drug to provide brand identification against a potential counterfit product. This identification may be done without destroying the tablet. The CTMD system can then be coated if desired to provide a barrier against the environment. The concept applies to capsules as well (CarreKap is the intended commercial name for this embodiment).

The CTMD system can be used either for immediate release tablets or for sustained or controlled release tablets. It is very useful to handle potent (low dose) drugs. The drugs in the surface can be colored to identify the different dose strengths. All low dose drugs (less than 10 mg) are good candidates for the surface portion of the CTMD. For example, steroidal hormones such as testosterone, progesterone, and cortisone derivatives are good candidates. Synthetic hormones such as levothyroxine, which is present in more than 10 tablets strengths in the market, is also a good candidate. For example, levothyroxine can be formulated in the core tablet and the metabolite, liothyronine could be formulated in the mini-disks in different strengths.

### Benefits of the Multi-Dosage Drug/Multi-Drug Delivery System

The benefits of the multi-dosage/multi-drug delivery system are similar to the benefits of using a core tablet having a single concavity. The CTMD has the added benefits of enhancing the bonding between drug semisolid formulation and core tablet, and of allowing several doses or drugs in the same dosage forms without being in physical contact. This minimizes drug incompatibilities in the dosage form.

Benefit for the Patient (improve patient adherence): the system will provide a smaller dosage form to patients who take several drugs and have trouble adhering to the therapy. The CarreDisk allows for multidrug administration in tiny disks within one tablet, thus minimizing the number of tablets a patient has to take. For example, a patient that takes 4 drugs two times a day, could take two CTMDs instead of eight tablets in a day.

Improved Manufacturing Costs (cost reduction via cycle time reductions): for a one drug delivery, the system will reduce dramatically the cost of producing a tablet since the majority of core tablets will be placebo tablets and the cleaning validation efforts as well as the potential for contamination will be greatly minimized. This will reduce processing cycle times for producing the core placebo tablet which will help reduce the cost of the producing the medicine. Since the drug will be formulated in the liquid form, there is no drug powder in the solids processing rooms (powder blending, tablet compression or encapsulation) and thus, no need for in-process testing for drug content during the tableting process.

Improved Quality Compliance (PAT Application, PAT Enabled Dosage Form):

The drug spot(s) will be identifiable by the naked eye and by analytical instruments which will allow the implementation of Process Analytical Technologies (PAT) at line; potentially enabling 100% of the tablets produced to be inspected by PAT for drug identification and possibly drug content; suitable for IR measurements (infrared spectroscopy).

### Example 5

### Embossed Tablets

In another aspect, the inventive drug delivery system includes a molded tablet having a design embossed thereon rather than concavities. See FIGS. 5A-E. The active ingredient or drug is formulated with the design material and applied to the tablet with application of the design; i.e. drug in print.

### CONCLUSION

The invention described and exemplified herein represents a new concept of tablet formulation/drug delivery system for delivery of low dose (i.e. potent) drugs, for delivery of multiple doses of a drug, or for delivery of multiple types of drugs. This concept leads to an overall improvement in quality in both the drug itself and in the manufacturing process.

All patents and publications mentioned in this specification are indicative of the levels of those skilled in the art to which the invention pertains. All patents and publications are herein incorporated by reference to the same extent as if each individual publication was specifically and individually indicated to be incorporated by reference. It is to be understood that while a certain form of the invention is illustrated, it is not intended to be limited to the specific form or arrangement herein described and shown. It will be apparent to those skilled in the art that various changes may be made without departing from the scope of the invention and the invention is not to be considered limited to what is shown and described in the specification. One skilled in the art will readily appreciate that the present invention is well adapted to carry out the objectives and obtain the ends and advantages mentioned, as well as those inherent therein. The tablets, drug delivery systems, methods, procedures, and techniques described herein are presently representative of the preferred embodiments, are intended to be exemplary and are not intended as limitations on the scope. Changes therein and other uses will occur to those skilled in the art which are encompassed within the spirit of the invention. Although the invention has been described in connection with specific, preferred embodiments, it should be understood that the invention as ultimately claimed should not be unduly limited to such specific embodiments. Indeed various modifications of the described modes for carrying out the invention which are obvious to those skilled in the art are intended to be within the scope of the invention.

What is claimed is:

1. A compressed, inactive tablet for carrying an active substance, the tablet comprising a concavity in a top surface configured for receiving the active substance.

2. The compressed, inactive tablet according to claim 1, formulated for immediate release of an active substance received in the concavity.

3. The compressed, inactive tablet according to claim 1, wherein the concavity is an indentation in the top surface to about 35% of a thickness of the tablet.

4. The compressed, inactive tablet according to claim 3, wherein the indentation is shaped to receive a predetermined volume of the active substance.

5. The compressed, inactive tablet according to claim 1, wherein the top surface of the tablet does not extend in height above edges of the concavity.

6. The compressed, inactive tablet according to claim 1, wherein the tablet has a diameter and the concavity has a diameter and the ratio of the tablet diameter to the concavity diameter is about 8:3.

7. A drug delivery system comprising:
a compressed, inactive tablet having a concavity in a top surface for receiving an active substance; and
an active substance deposited in the concavity.

8. The drug delivery system according to claim 7, wherein the active substance is a drug.

9. The drug delivery system according to claim 8, wherein the tablet is formulated for immediate release of the drug from the concavity.

10. The drug delivery system according to claim 8, wherein the drug is any one of a steroidal hormone, a synthetic hormone, and amlodipine besylate.

11. The drug delivery system according to claim 10, wherein the steroidal hormone is hydrocortisone and the synthetic hormone is levothyroxine.

12. The drug delivery system according to claim 8, wherein an amount of the drug deposited in the concavity ranges from about 1 to about 20 mg.

13. The drug delivery system according to claim 8, wherein an amount of the drug deposited in the concavity ranges from about 1 to about 5 mg.

14. The drug delivery system according to claim 8, wherein a concentration of the drug is about 33% w/v and could reach 50% or more.

15. The drug delivery system according to claim 8, wherein the tablet further comprises a pharmaceutically-acceptable coating for protecting the drug.

16. The drug delivery system according to claim 15, wherein the pharmaceutically-acceptable coating is one or more of hydroxypropyl methylcellulose (HPMC), polyvinylpyrrolidone (PVP), cellulose derivatives, starch derivatives, acrylates, acetates, gelatin, and shellac.

17. A method for preparing a drug delivery system, the method comprising:
selecting a drug for delivery;
selecting a polymer for dispersion of the drug;
preparing a liquid or semi-solid drug dispersion by dispersing the drug in the polymer; and
depositing a droplet of the drug dispersion in a concavity in a top surface of a compressed, inactive tablet such that the droplet solidifies to form a disc on the top surface of the tablet.

18. The method according to claim 17, further comprising selecting a temperature at which to deposit the droplet of the drug dispersion.

19. The method according to claim 17, wherein the selected drug has a therapeutic dose of 25 mg or less.

20. The method according to claim 17, wherein the selected polymer for dispersion is polyethylene glycol (PEG) or similar polymer with fatty or hydrophobic character.

21. The method according to claim 17, wherein the dispersing includes dispersing the drug in the polymer in an amount from about 1 mcg to about 10 mg.

22. The method according to claim 17, wherein the dispersing includes dispersing the drug in the polymer in an amount from about 1 mcg to about 5 mg.

23. The method according to claim 17, wherein volume of the droplet deposited ranges from about 0.25 μl to about 10 μl.

24. The method according to claim 17, further comprising coating the tablet with a pharmaceutically-acceptable coating to protect the drug.

25. The method according to claim 24, wherein the pharmaceutically-acceptable coating is one or more of hydroxypropyl methylcellulose (HPMC), polyvinylpyrrolidone (PVP), cellulose derivatives, starch derivatives, acrylates, acetates, gelatin, and shellac.

26. The method according to claim 17, wherein the drug selected is any one of a steroidal hormone, a synthetic hormone, and amlodipine besylate.

27. The method according to claim 26, wherein the steroidal hormone selected is hydrocortisone and the synthetic hormone selected is levothyroxine.

28. A drug delivery system prepared according to the method of claim 17, wherein the drug is any one of hydrocortisone, levothyroxine, and amlodipine besylate.

29. A compressed, inactive tablet for carrying at least one active substance, the tablet comprising:
a concavity in a top surface configured for receiving the at least one active substance; and
a plurality of concavities spaced apart and embedded within the concavity in the top surface, each of the plurality of concavities configured for receiving an active substance.

30. The compressed, inactive tablet according to claim 29, wherein the plurality of concavities includes three concavities.

31. The compressed, inactive tablet according to claim 29, wherein the tablet has a semi-solid formulation.

32. The compressed, inactive tablet according to claim 29, further comprising an identifier for facilitating detection.

33. The compressed, inactive tablet according to claim 32, wherein the identifier is ink or is embedded in ink.

34. The compressed, inactive tablet according to claim 29, wherein in the concavity in the top surface is an indentation in the top surface to about 35% of a thickness of the tablet.

35. The compressed, inactive tablet according to claim 33, wherein the top surface does not extend in height above edges of the concavity.

36. The compressed, inactive tablet according to claim 29, wherein the tablet has a diameter of about 8 mm and the concavity in the top surface has a diameter of about 3 mm.

37. The compressed, inactive tablet according to claim 29, wherein the tablet is shaped as a bullet having at least one curved end and an indentation for the concavity in the curved end or in a flat side.

38. The compressed, inactive tablet according to claim 29, wherein the tablet has a diameter and the concavity has a diameter and the ratio of the tablet diameter to the concavity diameter is about 8:3.

39. A drug delivery system comprising:
a compressed tablet having a concavity in a top surface for receiving at least one active sub stance;
a plurality of concavities spaced apart and embedded within the concavity in the top surface; and
an active substance deposited in the concavity in the top surface or an active substance deposited in at least one of the plurality of concavities.

40. The drug delivery system according to claim 39, wherein the active substance is a drug.

41. The drug delivery system according to claim 39, wherein the tablet has a semi-solid formulation.

42. The drug delivery system according to claim 40, wherein the drug is any one of a steroidal hormone, a synthetic hormone, and amlodipine besylate.

43. The drug delivery system according to claim 42, wherein the steroidal hormone is a cortisone derivative such as hydrocortisone and the synthetic hormone is levothyroxine.

44. The drug delivery system according to claim 40, wherein the tablet further comprises a pharmaceutically-acceptable coating for protecting the drug.

45. The drug delivery system according to claim 44, wherein the pharmaceutically-acceptable coating is one or more of hydroxypropyl methylcellulose (HPMC), polyvinylpyrrolidone (PVP), cellulose derivatives, starch derivatives, acrylates, acetates, gelatin, and shellac.

46. The drug delivery system according to claim 39, wherein the tablet is inactive.

47. The drug delivery system according to claim 46, wherein the active substance is deposited in the concavity in the top surface and each of the plurality of concavities is configured to support the active substance deposited in the concavity in the top surface.

48. The drug delivery system according to claim 47, wherein the tablet is formulated for immediate release of the active substance deposited in the concavity in the top surface.

49. The drug delivery system according to claim 39, wherein the active substance is a drug deposited in at least one of the plurality of concavities.

50. The drug delivery system according to claim 49, wherein the drug is formulated as a solid, a semi-solid, an emulsion, a suspension, a solution, a gel, or a fatty depot.

51. The drug delivery system according to claim 50, wherein the drug is a water-soluble drug or a water-insoluble drug.

52. The drug delivery system according to claim 51, wherein volume of the drug ranges from about 0.5 μl to about 500 μl.

53. The drug delivery system according to claim 39, wherein the plurality of concavities includes three concavities spaced apart from each other.

54. The drug delivery system according to claim 53, wherein an active substance is deposited in each of the three concavities.

55. The drug delivery system according to claim 54, wherein the active substance deposited in each of the three concavities is a drug.

56. The drug delivery system according to claim 55, wherein each of the three concavities includes a different dosage of the same drug.

57. The drug delivery system according to claim 55, wherein each of the three concavities includes a different drug.

58. The drug delivery system according to claim 39, wherein the tablet is formulated to include at least one active substance.

59. The drug delivery system according to claim 58, wherein the at least one active substance is a drug.

60. The drug delivery system according to claim 59, wherein the drug is levothryroxine and an active substance deposited in the concavity in the top surface is liothyronine.

61. The drug delivery system according to claim 59, wherein the drug is levothryroxine and an active substance deposited in each of the three concavities is liothyronine in different dosages.

62. A tablet for a drug delivery system comprising an active substance and/or an identifier embedded on a surface of the tablet.

63. The tablet according to claim 62, wherein the active substance is a drug embedded on the surface of the tablet.

64. The tablet according to claim 62, wherein the identifier is embedded in ink imprinted on the surface of the tablet.

65. A method for delivering at least one active substance to a subject in need thereof from a surface of a tablet carrier, the method comprising:
providing a compressed tablet having a concavity in a top surface for receiving at least one active substance and at least one active substance deposited in the concavity; and
administering the tablet to the subject in need thereof.

66. The method according to claim 65, wherein the providing includes providing a compressed, inactive tablet.
